(19) United States
(12) Patent Application Publication
(10) Pub. No.: US 2015/0125889 A1
(43) Pub. Date: May 7, 2015

(54) IN VITRO DETECTION OF MICROORGANISMS WITH AZOREDUCTASE ACTIVITY

(71) Applicant: BIOMÉRIEUX, Marcy L'Etoile (FR)

(72) Inventors: Arthur James, Cumbria (GB); Céline Roger-Dalbert, Quebec (CA); Claire Mercier, Lyon (FR); Sylvain Orenga, Neuville Sur Ain (FR)

(21) Appl. No.: 14/373,169

(22) PCT Filed: Jan. 18, 2013
(86) PCT No.: PCT/FR2013/050120

§ 371 Date: Jul. 18, 2014

(30) Foreign Application Priority Data
Jan. 19, 2012 (FR) 1250535

(51) Int. Cl. G01N 33/569

(52) U.S. Cl. CPC G01N 33/56961; G01N 33/56916; G01N 33/56911; G01N 33/56938; G01N 2333/90688
USPC 435/25

(57) ABSTRACT

The present invention relates to the use of at least one azo compound for detecting at least one microorganism in a sample. More precisely, the present invention relates to a process for detecting, in a biological sample, at least one microorganism with azoreductase activity, including the steps: placing the sample in contact with a reaction medium including at least one azo compound, incubating the reaction medium, and detecting the reduction of the azo compound by the microorganism, indicating the presence of the at least one microorganism.

The present invention relates to the field of microbiology. More specifically, it relates to a process for detecting microorganisms, in a sample, comprising a step consisting in detecting the reduction, by the said microorganisms, of an azo compound.

In general, the detection of microorganisms in a sample involves a step of placing the said sample in contact with a reaction medium and detecting changes in this reaction medium, which are markers of the presence of microorganisms. The microorganism detection method thus implemented should preferably be substantially independent of the nature of the sample in which the microorganisms are sought, and should have great sensitivity.

When the reaction medium is solid, the method consists of the direct observation of cells or the observation of the formation of colonies.

A further degree of complexity arises when it is desired to automate the detection.

A first method consists of observation of the reaction medium by nephelometry or turbidimetry, i.e. by revelation of the opacity of the reaction medium.

Other methods, via automated or non-automated biochemical routes, make use of detection by colorimetry or fluorimetry.

It is possible to detect the reaction between the sample and a reagent comprising a carbohydrate such as glucose, by means of a pH indicator.

It is possible to detect the reaction between the sample and a chromogenic or fluorogenic indicator of reduction or of oxidoreduction, as reported in patent EP 0 424 293 B1.

It is possible to use chromogenic or fluorescent enzymatic synthesis substrates (for a review see Orenga et al., 2009; J. Microbiol. Methods; 79(2):139-55). Such substrates are usually adapted to specific enzymatic activities and allow a targeted detection of microorganisms in clinical, food or environmental samples.

It is possible, in particular when the container of the reaction medium is a sealed container, to reveal a change in the pH or the CO₂ concentration, as reported in patent EP 0 790 299 B1.

In the light of this prior art, routes remain at the present time for searching for universal substrates, and/or substrates that allow a rapid response, and/or substrates that can be used with a complex sample such as a food matrix, and/or substrates that allow the detection of microorganisms which are difficult to detect. In this regard, the present invention relates to a novel method for detecting at least one microorganism with azoreductase activity, which may be present in a sample, comprising the steps consisting in:
placing the sample in contact with a reaction medium comprising at least one azo compound;
incubating the said reaction medium; and
detecting the reduction of the azo compound by the said at least one microorganism, indicating the presence of the said microorganism.

Azo compounds are molecules comprising one or more “azo” bonds, of the type R1-N═N—R2. They are essentially known as dyes, used in the textile, plastics, cosmetics and agrifood industries. It has been reported that microbial and mammalian enzymatic systems degrade azo compounds (Xu et al., 2010; Anaerobe, 16: 114-119). The presence of azoreductase activity in bacterial species of the human colon has even led to the synthesis of prodrugs comprising polymers bearing azo cross bonds (Chourasia & Kain, 2003; J. Pharm. Pharmacet. Sci., 6 (1): 33-66). Compounds comprising an azo bond have also been described as “quenchers”, i.e. they modify the fluorescence properties of a medium or of a fluorophore, as in patent application WO 2005/049 849. They may then be conjugated with biological molecules such as lipids, nucleic acids, peptides, proteins, etc. In this respect, azo derivatives are used for marking oligonucleotides, for the specific detection of sequences: the non-hybridized oligonucleotide is non-fluorescent; there is emission of fluorescence when it is hybridized on its complementary target sequence.

The Applicant describes herein a novel use of azo compounds, implemented in in vitro diagnostic methods, and in microbiological control methods, for example in the agrifood, pharmaceutical or cosmetological industry or in environmental control.

The definitions that follow are given for the purpose of facilitating the understanding of the present invention.

The term biological sample means an isolated small part or small amount of a species for analysis. This may be a human or animal clinical sample, derived from a withdrawal of biological liquid, or a food sample, derived from any type of food, a pharmaceutical or cosmetological product, or a sample from the environment for producing or processing foods or pharmaceutical or cosmetological products. This sample may thus be liquid or solid. Mention may be made, in a non-limiting manner, of a clinical sample of whole blood, serum, plasma, urine or faeces, samples collected from the nose, the throat, the skin, wounds or cerebrospinal fluid, a sample of food, water, drinks such as milk, a fruit juice, yoghourt, meat, eggs, vegetables, mayonnaise, cheese, fish, etc., a sample derived from feed intended for animals, especially such as a sample derived from animal or plant meal, or a surface or water control sample. In the case of the sample of food origin, it is also referred to as a food matrix.

This sample may be used in unmodified form or, prior to the analysis, may undergo a preparation of enrichment, dilution, extraction, concentration or purification type, according to methods known to those skilled in the art.

For the purposes of the present invention, the term “microorganism” covers bacteria, yeasts, moulds and, more generally, organisms which are generally unicellular, invisible to the naked eye, and may be multiplied and manipulated in the laboratory. Gram-negative bacteria that may be mentioned include bacteria of the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Branhamella, Neisseria, Burkholderia, Citrobacter, Hafnia, Edwardsiella, Aeromonas, Moraxella, Pasteurella, Providencia, Actinobacillus, Alcaligenes, Bordetella, Cedecea, Erwinia, Pantoea, Ralstonia, Stenotrophomonas, Xanthomonas and Legionella.

Gram-positive bacteria that may be mentioned include bacteria of the following genera: Aerococcus, Enterococcus, Streptococcus, Staphylococcus, Bacillus, Lactobacillus, Listeria, Clostridium, Gardnerella, Kocuria, Lactococcus, Leuconostoc, Micrococcus, Falkamia, Gemella, Pediococcus, Mycobacterium and Corynebacterium.

Yeasts that may be mentioned include yeasts of the following genera: Candida, Cryptococcus, Saccharomyces and Trichosporon.

Moulds that may be mentioned include moulds of the following genera: Aspergillus, Fusarium, Geotrichum and Penicillium.

The term reaction medium means a medium comprising all the elements necessary for the expression of a metabolism and/or growth of microorganisms. The reaction medium may be solid, semi-solid or liquid. The term “solid medium” means, for example, a gelled medium. Agar is the conventional gelling agent in microbiology for culturing microorganisms, but it is possible to use gelatin, agarose or other natural or artificial gelling agents. A certain number of preparations are commercially available, for instance Columbia agar, trypcase-soya agar, Mac Conkey agar, Mueller Hinton agar or, more generally, those described in the Handbook of Microbiological Media (CRC Press).

The reaction medium may comprise one or more elements in combination, such as amino acids, peptones, carbohydrates, nucleotides, minerals, vitamins, etc. The medium may also comprise a dye. As a guide, dyes that may be mentioned include Evans blue, neutral red, sheep blood, horse blood, an opacifier such as titanium oxide, nitroaniline, malachite green, brilliant green, one or more metabolic indicators, one or more metabolic regulators, etc.

The reaction medium may be a revelation medium or a culture and revelation medium. In the first case, the culturing of microorganisms is not necessary or is performed before seeding, and, in the second case, the detection and/or characterization medium also constitutes the culture medium.

The reaction medium may comprise one or more selective agents. The term “selective agent” means any compound that is capable of preventing or slowing down the growth of a microorganism other than the target microorganism. Without being limiting, a concentration of between 0.01 mg/l and 5 g/l is particularly suitable for use in the present invention.

Selective agents that may be mentioned include antibiotics, antifungal agents, bile salts, crystal violet, basic fuchsin, brilliant green, etc. The term “antibiotic” means any compound that is capable of preventing or slowing down the growth of a bacterium. They especially belong to the groups of beta-lactamines, glycopeptides, aminosides, polypeptides, sulfamides or quinolones.

The term “antifungal agent” means any compound that is capable of preventing or slowing down the growth of a yeast or mould. As a guide, mention may be made especially of amphotericin B, fluconazole, itraconazole, voriconazole or cycloheximide.

The term “azo compound” means any molecule comprising at least one azo group, i.e. at least one bond of the type R₁—N═N—R₂.

The term “azoreductase” means any enzyme, irrespective of its structure and its classification, which is capable of reducing an azo function.

The term “reduction” means in practice the reduction of the azo double bond (—N═N—).

This reduction may be total and lead to formation of two amine residues (—NH₂), but it may be partial and lead to a partially reduced bond, for example —NH—NH—.

The term “substrate or chromogenic substrate” means a compound enabling the detection of enzymatic or metabolic activity by means of a directly or indirectly detectable signal. Preferentially, the said enzymatic or metabolic activity is that of a microorganism. The term “chromogenic substrate” means a compound enabling the detection of enzymatic or metabolic activity by means of the variation of an optical signal such as an absorbance and/or fluorescence variation. For a direct detection, this substrate may be linked to a part acting as a fluorescent or coloured marker (Orenga et al., 2009; J. Microbiol. Methods; 79(2):139-55). For an indirect detection, the reaction medium according to the invention may in addition comprise a pH indicator, which is sensitive to the variation in pH induced by the consumption of the substrate and revealing the metabolism of the target microorganisms. The said pH indicator may be a chromophore or a fluorophore. Examples of chromophores that will be mentioned include bromocresol purple, bromothymol blue, neutral red, aniline blue and bromocresol blue. The fluorophores comprise, for example, 4-methylumbelliferone, hydroxycoumarin derivatives, fluorescein derivatives or resorufin derivatives.

As a guide, the enzymatic activities targeted by the chromogenic substrates may belong to the group of hydrolases, preferentially to the group of osidases, esterases or peptidases. Preferentially, the enzymatic activities targeted by the chromogenic substrates are chosen from: glucuronidase, glucosidase, galactosidase, esterase, sulfatase and deaminase. Needless to say, the azo compounds used in the process according to the invention correspond to substrates that detect azoreductase activity. The term “incubating” means bringing to and maintaining at, for between 5 minutes and 48 hours, preferentially between 4 and 24 hours and more preferentially between 16 and 24 hours, a suitable temperature, generally between 20 and 50° C. and preferentially between 30 and 40° C.

The term “detecting” means discerning with the naked eye or using an optical machine the existence of growth and/or activity of the target bacteria. Advantageously, when the medium used comprises a chromogenic substrate, the detection may also allow characterization of the target microorganisms.

The term “quencher” means an element for reducing the fluorescence intensity of a given substance. A quencher may be defined as an extinctor, by absorption of the excitation or emission energy. Quenching may then be defined as an extinction or suppression of the excitation or emission wavelength, or by the substitution of a group on a molecule, the said substitution inducing a change in the excitation capacity of the electrons. The term “group” means nitrogenous, hydroxyl, thiol, carbon-based, methyl, propyl, butyl, phenyl, etc. groups or residues.

The present invention relates to a process for detecting, in a biological sample, at least one microorganism with azoreductase activity, comprising the steps consisting in:
a) placing the sample in contact with a reaction medium comprising at least one azo compound,
b) incubating the said reaction medium, and
c) detecting the reduction of the azo compound by the said microorganism, indicating the presence of the said at least one microorganism.

Advantageously, step c) also allows counting of the microorganisms.

Advantageously, the process according to the invention allows the characterization (or identification) of at least one group of microorganisms. In other words, it makes it possible both to detect and to determine which group of microorganisms is detected.

The majority of azo compounds are coloured in the oxidized state and colourless in the reduced state. The reduction of azo compounds may lead to the formation of a fluorescent compound. Thus, preferentially, the reduction of the azo compound is detected by measuring a variation in absorbance or fluorescence. Preferentially, the reduction of the azo compound is detected by the disappearance of a coloration and/or by the appearance of fluorescence.

According to the present invention, the incubation step may be performed aerobically or anaerobically, in other words in the presence or absence of oxygen. Specifically, the activity of the microorganisms, detected according to the present invention, is linked to the aerobic and/or anaerobic respiratory metabolism of the microorganisms. The majority of microorganisms thus have this type of activity.

According to a particular embodiment, the azo compounds used in the process according to the invention may be coupled to a fluorophore whose emission wavelength or excitation wavelength is quenched by the coloration of the azo compound.

According to another particular embodiment, the azo compounds used in the process according to the invention can quench the fluorescence of another molecule.

To summarize, it is possible:
to detect the disappearance of coloration of the azo compound;
to detect the natural fluorescence of a reduction product; or
to detect a fluorophore coupled with the azo compound or a fluorophore corresponding to a different molecule, the fluorescence appearing by disappearance of the quenching phenomenon.

Among the preferred fluorophores, mention will be made in a non-limiting manner of coumarins, among which are AMC (7-amino-4-methylcoumarin), 4-MU (4-methylumbelliferone), fluorescein derivatives, etc.

The coupling between the fluorophore and the azo compound may be intramolecular coupling or intermolecular coupling. The term “intramolecular coupling” means that the fluorophore is covalently bonded to the azo compound. For example, the azo compound may be coupled with a fluorophore, which is capable of being excited or of emitting at the absorbance wavelength of the oxidized azo compound. The coloured oxidized compound then masks the fluorescence. After reduction, the compound is decolourized and reveals the fluorescence by disappearance of the intramolecular quenching phenomenon.

The term “intermolecular coupling” means that the fluorophore and the azo compound are two different chemical molecules. The reduction of the azo compound then brings about the possible appearance of fluorescence by disappearance of the intermolecular quenching phenomenon.

Advantageously, the reaction medium used in the process according to the invention also comprises at least a second substrate that is capable of detecting enzymatic activity. Preferentially, the said substrate is a chromogenic substrate, i.e. the metabolism of the said substrate produces a coloration or a fluorescence. Preferentially, the said enzymatic activity is different from the azoreductase activity.

The process according to the invention is of particular interest for samples of food type. Currently, the detection of the total flora by means of a commercially available kit uses three fluorescent substrates and permits reading of the result within 48 hours. The drawback is that certain food matrices have crossed reactions with this kit. In other words, a non-specific enzymatic reaction linked to the food itself leads to false-positive results. The tests performed with azo compounds show that the food matrices that have the largest number of false-positive results in this test for detecting the total flora do not have any background noise with the azo compound. Thus, advantageously, the process according to the invention may be performed in a solid container such as a microplate, microtube, microcrucible, capillary tube, or a multiwell card such as the Vitek® card or the Tempo® card. Preferentially, the reaction card is a Vitek® card or a Tempo® card.

Finally, the invention also relates to the use of at least one azo compound for detecting at least one microorganism included in a sample.

The examples developed below are aimed at facilitating the understanding of the invention. They are given for explanatory purposes and shall not limit the scope of the invention.

### EXAMPLE 1

### Tests of Reduction Over 24 Hours at 35° C. of Compounds Bearing an “Azo” Function by 10 Strains Representing 10 Species of Microorganisms

Reduction of the “azo” function of the substrates listed in Table I by the species listed in Table II, each species being represented by a strain, is tested in a microtitration plate.

TABLE I

| | |
|---|---|
| Methyl red | 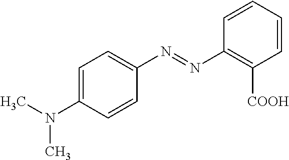 [SMILES: CN(C)c1ccc(/N=N/c2ccccc2C(=O)O)cc1] |
| 3-(4-Hydroxyphenylazo)benzoic acid, sodium salt | 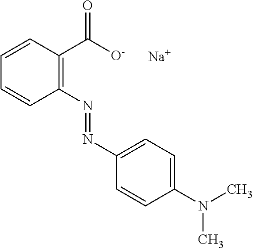 [SMILES: CN(C)c1ccc(/N=N/c2ccccc2C(=O)[O-])cc1 ; [Na+]] |
| 2-(4-Acetoxyphenylazo)benzoic acid | 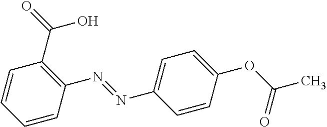 [SMILES: CC(=O)Oc1ccc(/N=N/c2ccccc2C(=O)O)cc1] |
| 3-(4-Hydroxyphenylazo)benzoic acid | 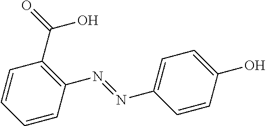 [SMILES: O=C(O)c1ccccc1/N=N/c1ccc(O)cc1] |
| 2-Hydroxy-3,5- dibromophenylazo)benzoic acid | 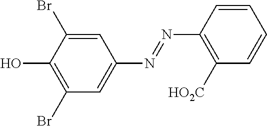 [SMILES: O=C(O)c1ccccc1/N=N/c1cc(Br)c(O)c(Br)c1] |
| 2-(4-Hydroxy-3,5- dichlorophenylazo)benzoic acid | 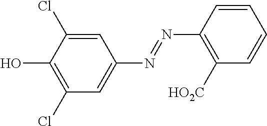 [SMILES: O=C(O)c1ccccc1/N=N/c1cc(Cl)c(O)c(Cl)c1] |
| Methyl orange | 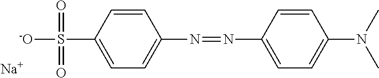 |
| Orange II | 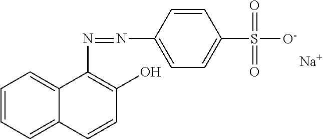 [SMILES: O=S(=O)([O-])c1ccc(N=Nc2c(O)ccc3ccccc23)cc1 ; [Na+]] |
| 4-Aminoazobenzene-3,4′- disulfonic acid | 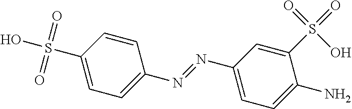 [SMILES: Nc1ccc(/N=N/c2ccc(S(=O)(=O)O)cc2)cc1S(=O)(=O)O] |
| Sunset Yellow | 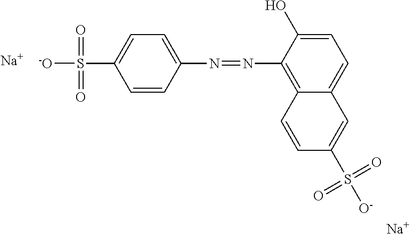 |
| BF38 = 4″-Quinoline-4′-styryl- azo-2-Hydroxynaphthol | 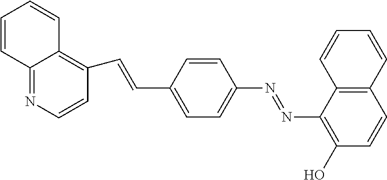 [SMILES: Oc1ccc2ccccc2c1/N=N/c1ccc(/C=C/c2ccnc3ccccc23)cc1] |
| Orange I | 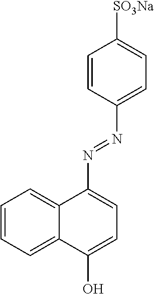 |
| BF45 = 2-Phenyl- benzothiazole-4′-azo-4″- hydroxybenzene-3″-carboxylic acid | 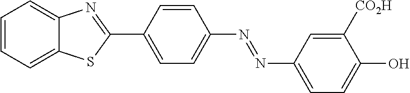 [SMILES: O=C(O)c1cc(/N=N/c2ccc(-c3nc4ccccc4s3)cc2)ccc1O] |
| Bordeaux B | 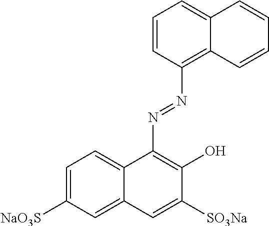 |
| Ponceau 2G | 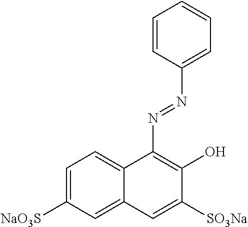 |
| Tartrazine | 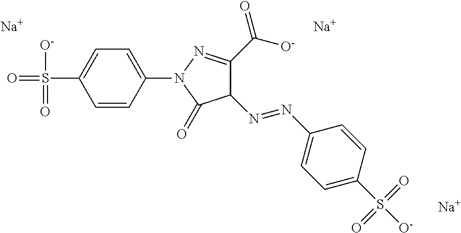 [SMILES: [Na+] ; O=C([O-])C1=NN(c2ccc(S(=O)(=O)[O-])cc2)C(=O)C1/N=N/c1ccc(S(=O)(=O)[O-])cc1 ; [Na+] ; [Na+]] |

TABLE II

Escherichia coli
Cronobacter muytjensii
Acinetobacter baumanii
Pseudomonas aeruginosa
Staphylococcus aureus
Staphylococcus epidermidis
Kocuria rosea
Enterococcus faecalis
Candida albicans
Geotrichum candidum

The reaction medium consists of:
Trypcase soya broth
Substrate 35 mg/l
0.5 MacFarland inoculum

The reduction of the substrate and the growth of the microorganisms are monitored using an Infinite® M200 Tecan microplate reader at 35° C. over 24 hours.

The growth is monitored by the absorbance at 660 nm and the reduction of the substrates by absorbance and fluorescence at the specific wavelengths indicated in Table III.

TABLE III

| | Iₘₐₓ | Iₘₐₓ | Iₘₐₓ |
|---|---|---|---|
| | Absorption | excitation | emission |
| | (nm) | (nm) | (nm) |
| Methyl red | 435 | 250 | 395 |
| 3-(4-Hydroxyphenylazo)benzoic | 435 | 250 | 395 |
| acid, sodium salt | | | |
| 2-(4-Acetoxyphenylazo)benzoic | 345 | 250 | 395 |
| acid | | | |
| 3-(4-Hydroxyphenylazo)benzoic | 355 | 250 | 394 |
| acid | | | |
| 2-(4-Hydroxy-3,5- | 410 | 250 | 396 |
| dibromophenylazo)benzoic acid | | | |
| 2-(4-Hydroxy-3,5- | 355 | 250 | 395 |
| dichlorophenylazo)benzoic acid | | | |
| Methyl orange | 470 | 255 | 337 |
| Orange II | 490 | 348 | 446 |
| 4-Aminoazobenzene-3,4′-disulfonic | 380 | 329 | 437 |
| acid | | | |
| Sunset Yellow | 485 | 367 | 476 |
| BF38 | 390 | 341 | 505 |
| Orange I | 483 | 320 | 473 |
| BF45 | 392 | 337 | 421 |
| Bordeaux B | 530 | 320 | 445 |
| Ponceau 2G | 496 | 320 | 486 |
| Tartrazine | 435 | 255 | 336 |

The substrate is considered as reduced by the microorganisms when the substrate is decolourized (monitored by the decrease in absorbance) (Decol) and/or when the fluorescence of the substrate is multiplied by 2 relative to the base fluorescence of a substrate-free control (Fluo). The growth is indicated by Co. The symbol “+” means good growth/decolourization/significant increase in fluorescence. The symbol “+/−” means difficult growth. The symbol “−” means that there is no growth/decolourization/significant increase in fluorescence. RED indicates whether the substrate is reduced (Y) or not (N).

TABLE IV

| | Gram− species | | | | | | | | | | | | | | | |
|---|---|---|---|---|---|---|---|---|---|---|---|---|---|---|---|---|
| | E. coli | | | | E. muytjensii | | | | A. baumannii | | | | P. aeruginosa | | | |
| | Co | Decol | Fluo | RED | Co | Decol | Fluo | RED | Co | Decol | Fluo | RED | Co | Decol | Fluo | RED |
| Methyl red | + | + | + | Y | + | + | + | Y | + | + | + | Y | + | +/− | + | Y |
| 2-(4-hydroxyphenylazo)benzoic acid, sodium | + | + | + | Y | + | + | + | Y | + | + | + | Y | + | +/− | + | Y |
| salt | | | | | | | | | | | | | | | | |
| 2-(4-acetoxyphenylazo)benzoic acid | + | + | + | Y | + | + | + | Y | + | + | + | Y | + | − | − | N |
| 2-(4-hydroxyphenylazo)benzoic acid | + | + | + | Y | + | + | + | Y | + | + | + | Y | + | +/− | + | Y |
| 2-(4-hydroxy-3,5-dibromophenylazo)benzoic | + | − | + | Y | + | + | + | Y | + | − | + | Y | + | − | − | N |
| acid | | | | | | | | | | | | | | | | |
| 2-(4-hydroxy-3,5-dichlorophenylazo)benzoic | + | + | + | Y | + | + | + | Y | + | + | + | Y | + | − | + | Y |
| acid | | | | | | | | | | | | | | | | |
| Methyl orange | + | − | − | N | + | − | − | N | + | − | − | N | + | − | − | N |
| Orange II | + | − | − | N | + | − | − | N | + | − | − | N | + | − | − | N |
| 4-aminoazobenzene-3,4′-disulfonic acid | + | − | − | N | + | − | − | N | + | − | − | N | + | − | − | N |
| Sunset Yellow | + | − | − | N | + | − | − | N | + | − | − | N | + | − | − | N |
| BF45 | + | − | + | Y | + | − | − | N | + | − | + | Y | + | − | − | N |
| Orange I | + | + | − | Y | + | + | − | Y | + | +/− | − | Y | + | − | − | N |
| BF38 | + | + | − | Y | + | + | − | Y | + | − | − | N | + | + | − | Y |
| Bordeaux B | + | − | − | N | + | − | − | N | + | − | − | N | + | − | − | N |
| Ponceau 2G | + | − | − | N | + | − | − | N | + | − | − | N | + | − | − | N |
| Tartrazine | + | − | − | N | + | − | − | N | + | − | − | N | + | − | − | N |
| | Gram+ species | | | | | | | | | | | | | | | |
| | S. aureus | | | | S. epidermidis | | | | E. faecalis | | | | K. rosea | | | |
| | Co | Decol | Fluo | RED | Co | Decol | Fluo | RED | Co | Decol | Fluo | RED | Co | Decol | Fluo | RED |
| Methyl red | + | + | + | Y | + | + | + | Y | + | + | + | Y | +/− | +/− | + | Y |
| 2-(4-hydroxyphenylazo)benzoic acid | + | + | + | Y | + | + | + | Y | + | + | + | Y | − | +/− | + | Y |
| sodium salt | | | | | | | | | | | | | | | | |
| 2-(4-acetoxyphenylazo)benzoic acid | + | + | + | Y | + | + | + | Y | + | + | + | Y | − | − | − | NA |
| 2-(4-hydroxyphenylazo)benzoic acid | + | + | + | Y | + | + | + | Y | + | + | + | Y | +/− | − | + | Y |
| 2-(4-hydroxy-3,5-dibromophenylazo)benzoic | + | + | + | Y | + | +/− | + | Y | + | + | + | Y | − | − | − | NA |
| acid | | | | | | | | | | | | | | | | |
| 2-(4-hydroxy-3,5-dichlorophenylazo)benzoic | + | + | + | Y | + | + | + | Y | + | + | + | Y | − | − | − | NA |
| acid | | | | | | | | | | | | | | | | |
| Methyl orange | + | − | − | N | + | − | − | N | + | + | + | Y | − | − | − | NA |
| Orange II | + | − | − | N | + | − | − | N | + | + | + | Y | − | − | − | NA |
| 4-aminoazobenzene-3,4′-disulfonic acid | + | − | − | N | + | − | − | N | + | + | + | Y | − | − | − | NA |
| Sunset Yellow | + | − | − | N | + | − | − | N | + | + | + | Y | − | − | − | NA |
| BF45 | + | + | + | Y | + | − | + | Y | + | + | + | Y | + | − | + | Y |
| Orange I | + | + | − | Y | + | + | − | Y | + | + | − | Y | − | − | − | NA |
| BF38 | + | + | − | Y | + | + | − | Y | + | + | − | Y | − | − | − | NA |
| Bordeaux B | + | − | − | N | + | − | − | N | + | + | + | Y | − | − | − | NA |
| Ponceau 2G | + | − | − | N | + | − | − | N | + | + | − | Y | − | − | − | NA |
| Tartrazine | + | − | − | N | + | − | − | N | + | + | +/− | Y | − | − | − | NA |

| Yeast species | |
|---|---|
| C. albicans | G. candidum |

| | Co | Decol | Fluo | RED | Co | Decol | Fluo | RED |
|---|---|---|---|---|---|---|---|---|
| Methyl red | + | + | + | Y | +/− | +/− | + | Y |
| 2-(4-hydroxyphenylazo)benzoic acid, sodium salt | + | − | − | N | + | + | + | Y |
| 2-(4-acetoxyphenylazo)benzoic acid | + | − | − | N | + | + | + | Y |
| 2-(4-hydroxyphenylazo)benzoic acid | + | − | − | N | +/− | − | − | N |
| 2-(4-hydroxy-3,5-dibromophenylazo)benzoic acid | + | − | − | N | + | − | + | Y |
| 2-(4-hydroxy-3,5-dichlorophenylazo)benzoic acid | + | − | − | N | + | + | + | Y |
| Methyl orange | + | − | − | N | + | − | − | N |
| Orange II | + | − | − | N | +/− | − | − | N |
| 4-aminoazobenzene-3,4′-disnlfonic acid | + | + | + | Y | + | − | − | N |
| Sunset Yellow | + | + | + | Y | + | − | − | N |
| BF45 | + | − | − | N | + | − | − | N |
| Orange I | + | − | − | N | +/− | − | − | N |
| BF38 | + | − | − | N | + | − | − | N |
| Bordeaux B | + | − | − | N | +/− | − | − | N |
| Ponceau 2G | + | − | − | N | +/− | − | − | N |
| Tartrazine | + | − | − | N | +/− | − | − | N |

The results reported in Table IV show that the microorganisms can reduce “azo” molecules if there is growth. Certain molecules can be reduced by all the microorganisms tested (examples: Methyl red) and others are reduced specifically by only one species (example: Methyl orange reduced only by E. faecalis). Consequently, the azoreductase activity may be used as a means for detecting microorganisms, either of a target group, or universally, depending on the choice of the azo compound. In the cases of Orange I, Ponceau 2G and BF 38, the decolourization of the substrate makes it possible to visualize an increase in fluorescence. This fluorescence is that of the microorganisms. In other words, it is intrinsic fluorescence that exists for the substrate-free controls. It is quenched by the coloration of the starting substrate and revealed gradually as the substrate decolourizes by reduction. In this case, in Table IV, the fluorescence is noted “−” since it is not that of the substrate. It is for this reason that cases of decolourization without an increase in fluorescence are observed (Table IV).

In conclusion, it is possible to detect the presence of microorganisms by using an “azo” substrate.

### EXAMPLE 2

### Tests of Reduction Over 24 Hours at 35° C. of 3 Compounds Bearing an “Azo” Function by 45 Strains Representing 25 Species of Microorganisms

The reduction of the “azo” function of the substrates listed in Table V by the 25 species listed in Table VI, each being represented by one or two strains, is tested in a microtitration plate.

TABLE V

3-(4-Hydroxyphenylazo)benzoic
acid, sodium salt
Tartrazine
Orange II

TABLE VI

| | Number of strains |
|---|---|
| Gram− species | |
| Escherichia coli | 2 |
| Cronobacier muytjensii | 2 |
| Cronobacter sakazakii | 2 |
| Enterobacter cloacae | 2 |
| Acinetobacter baumanii | 2 |
| Pseudomonas aeruginosa | 2 |
| Pseudomonas fluorescens | 2 |
| Pseudomonas putida | 2 |
| Proteus vulgaris | 2 |
| Citrobacter koseri | 1 |
| Gram+ species | |
| Staphylococcus aureus | 2 |
| Staphrhcoccus epidermidis | 2 |
| Staphylococcus saprophyticus | 2 |
| Staphylococcus hominis | 1 |
| Staphylococcus haemolyticus | 1 |
| Kocuria rosea | 2 |
| Enterococcus faecalis | 2 |
| Enterococcus faecium | 2 |
| Streptococcus agalactiae | 1 |
| Streptococcus pyogenes | 1 |
| Yeast species | |
| Candida albicans | 2 |
| Candida glabrata | 2 |
| Geotrichum candidum | 2 |
| Geotrichum capitatum | 2 |
| Saccharomyces cerevisiae | 1 |

The reaction medium consists of:
Trypcase soya broth
Substrate 35 mg/l
0.5 MacFarland inoculum

The reduction of the substrate and the growth of the microorganisms are monitored using an Infinite® M200 Tecan microplate reader at 35° C. over 24 hours.

The growth is monitored by the absorbance at 660 nm, and the reduction of the substrates by absorbance and fluorescence at the wavelengths indicated in Table III above.

The substrate is considered as reduced by the microorganisms when the fluorescence is multiplied by 2 relative to the base fluorescence of the reaction medium. The strains that reduce the substrate are marked with a “+” and the strains that do not reduce the substrate are marked with a “−”.

TABLE VII

| | 3-(4- | | |
|---|---|---|---|
| | Hydroxyphenylazo)- | | |
| | benzoic acid, | | |
| | sodium salt | Tartrazine | Orange II |
| Gram− species | | | |
| Escherichia coli | + | − | − |
| Escherichia coli | + | − | − |
| Cronobacter muytjensii | + | − | − |
| Cronobacter muytjensii | + | − | − |
| Cronobacter sakazakii | + | − | − |
| Cronobacter sakazakii | + | − | − |
| Enterobacter cloacae | + | − | − |
| Enterobacter cloacae | + | − | − |
| Acinetobacter baumanii | + | − | − |
| Acinetobacter baumanii | + | − | − |
| Pseudomonas aeruginosa | + | − | − |
| Pseudomonas aeruginosa | + | − | − |
| Pseudomonas fluorescens | + | − | − |
| Pseudomonas fluorescens | + | − | − |
| Pseudomonas putida | + | − | − |
| Pseudomonas putida | + | + | − |
| Proteus vulgaris | + | − | − |
| Proteus vulgaris | | − | − |
| Citrobacter koseri | + | − | − |
| Gram+ species | | | |
| Staphylocossus aureus | + | − | − |
| Staphylocossus aureus | + | − | − |
| Staphylococcus epidermidis | + | − | − |
| Staphylococcus epidermidis | + | − | − |
| Staphylococcus | + | − | − |
| saprophyticus | | | |
| Staphylococcus | + | − | − |
| saprophyticus | | | |
| Staphylococcus hominis | + | − | − |
| Staphylococcus | + | − | − |
| haemolyticus | | | |
| Kocuria rosea | + | − | − |
| Kocuria rosea | + | − | − |
| Enterococcus faecalis | + | + | + |
| Enterococcus faecalis | + | + | + |
| Enterococcus faecium | + | − | − |
| Enterococcus faecium | + | − | − |
| Streptococcus agalactiae | + | − | − |
| Streptococcus agalactiae | + | − | − |
| Streptococcus pyogenes | + | − | − |
| Yeast species | | | |
| Candida albicans | + | − | − |
| Candida albicans | − | − | − |
| Candida glabrata | + | − | − |
| Candida glabrata | − | − | − |
| Geotrichum candidum | + | − | − |
| Geotrichum candidum | − | − | − |
| Geotrichum capitatum | + | − | − |
| Geotrichum capitatum | + | − | − |
| Saccharomyces cerevisiae | − | − | − |

The results reported in Table VII show that 3-(4-hydroxyphenylazo)benzoic acid, sodium salt can be reduced by all the bacterial species tested and also by certain yeasts, whereas Tartrazine and Orange II are reduced mainly by E. faecalis.

In conclusion, the “azo” molecules may be used either for detecting the presence of any microbial genus, or for detecting the presence of specific microorganisms, or for differentiating or characterizing specific microorganisms.

### EXAMPLE 3

### Test of Reduction of Methyl Red by Food Matrices

Nine food matrices, some of which have high enzymatic activity (for which the use of Tempo®TVC is not recommended), listed in Table VIII, are tested, in a microtitration plate, in the presence of 35 mg/l of Methyl red in TSB medium to which is added an antimicrobial cocktail combining 200 mg/l of chloramphenicol and 6 mg/l of gentamycin to inhibit the growth of bacteria and fungi.

The matrices are tested at different concentrations: 1/400, 1/4000 and 4/40 000. To do this, 10 g of matrices are weighed out and dispersed by stomaching in 90 ml of Tryptone-salt solution, which corresponds to a 1/10 dilution, from which the test dilutions are made.

The reduction of the substrate and the growth of the microorganisms are monitored using an Infinite® M200 Tecan microplate reader at 35° C. over 24 hours.

The reduction of the Methyl red is monitored by reading the fluorescence with the wavelength pair 250 nm/395 nm. The potential growth of microorganisms is monitored by reading the absorbance at 660 nm. The symbol “−” means that there is no growth or increase in fluorescence of the medium indicating a reduction of the substrate over 24 hours.

In parallel, the same samples with antimicrobial cocktail are tested with the product Tempo®TVC (bioMérieux, France), and on antibiotic-free Columbia agar medium.

The Tempo®TVC medium is taken up in 3.9 ml of water+antimicrobial cocktail to which are added 100 μl of the solution of 1/10 food matrix sample. This suspension is transferred into the Tempo® card using the Tempo® Filler, it is incubated for 40-48 hours at 30±1° C. and the fluorescence is observed with the Tempo® Reader.

100 μl of the solution of 1/10 food matrix sample, corresponding to the amount of sample contained in the 1/400 concentration, are spread on the agar medium. The starting solution of 1/10 food matrix sample is successively diluted twice, and 100 μl of each solution corresponding to the amounts of sample contained in the 1/4000 and 1/40 000 concentrations tested previously are then plated out.

TABLE VIII

| | Tempo | Culture | 660 nm |
|---|---|---|---|
| Mussels + ATB | + | − | − |
| Pink shrimps + ATB | − | − | − |
| Raw calf liver + ATB | + | + | − |
| Fresh tagliatelle + ATB | − | − | − |
| Avocado + ATB | + | − | − |
| Melon + ATB | − | − | − |
| Salami + ATB | − | − | − |
| Brie de Meaux AOC cheese + ATB | + | + | − |
| Microfiltered whole milk + ATB | − | − | − |

Table VIII indicates the growth (or absence of growth) of microorganisms in the various samples detected by Tempo® TVC (Tempo), by culturing on Columbia agar (Culture) and by reading the optical density at 660 nm. Colonies are observed for the samples of calf liver and of brie de Meaux AOC cheese, whereas no growth is observed for these samples on reading at 660 nm. This difference may be due to the fact that the sample is maintained in an environment containing an antimicrobial cocktail, whereas the culture medium on which the sample is seeded lacks the antimicrobial cocktail.

TABLE IX

| | Increase in fluorescence |
|---|---|
| | over 24 hours |
| Mussels + ATB | − |
| Pink shrimps + ATB | − |
| Raw calf liver + ATB | − |
| Fresh tagliatelle + ATB | − |
| Avocado + ATB | − |
| Melon + ATB | − |
| Salami + ATB | − |
| Brie de Meaux AOC cheese + ATB | − |
| Microfiltered whole milk + ATB | − |

Table IX indicates the absence of increase of fluorescence (−) of the medium containing the food matrix in the absence of growth of microorganisms, thus showing that there is no reduction of the Methyl red by the food matrix.

The results reported in Table IX indicate that the matrices tested do not express any azoreductase activity capable of degrading Methyl red.

These results as a whole indicate that the matrices: mussels, calf liver, avocado and brie de Meaux AOC cheese emit fluorescence in the presence of the sample and of the TVC medium, in the absence of growth of microorganisms, whereas this is not the case with the Methyl red substrate. It may therefore be concluded that no non-microbial activity for the azoreduction of Methyl red by the food matrices interferes with the detection of the microbial azoreductase activity in the foods.

### EXAMPLE 4

### Test of Reduction of 3-(4-hydroxyphenyl)azobenzoic Acid by Microorganisms Contained in Food Matrices

Twelve food matrices are tested by 2 media: the commercial medium Tempo® TVC and the Azo medium manufactured in the laboratory, which contains the “azo” substrate 3-(4-hydroxyphenyl)azobenzoic acid (143.6 mg/L) coupled to 7-amino-4-methylcoumarin (30.8 mg/L) in trypcase soya broth. This example uses intermolecular coupling between an azo substrate and a fluorescent molecule. The red-coloured oxidized substrate 3-(4-hydroxyphenyl)azobenzoic acid quenches the fluorescence of 7-amino-4-methylcoumarin. Once reduced, the 3-(4-hydroxyphenyl)azobenzoic acid becomes colourless and allows the fluorescence of the 7-amino-4-methylcoumarin to appear. The 2 media are prepared with and without antimicrobial cocktail combining 500 mg/l of chloramphenicol and 10 mg/l of gentamycin to inhibit the growth of bacteria and fungi.

The matrices are tested at different concentrations: 1/400, 1/4000 and 1/40 000. To do this, 10 g of matrices are weighed out and dispersed by stomaching in 90 ml of Tryptone-salt solution, which corresponds to a 1/10 dilution, from which the test dilutions are prepared.

The product Tempo®TVC is taken up in 3.9 mL of water and the Azo medium is divided into 3.9 mL aliquots. 100 μL of matrix sample are added to each medium. The Tempo® cards are filled and incubated at 30° C., and read after 48 hours by the Tempo® Reader.

In parallel, counting on PCA medium, which is the reference method, is performed on the basis of CFU/g (CFU means colony-forming units, which is the counting unit known to those skilled in the art).

TABLE X

| | Growth on PCA | Growth on PCA |
|---|---|---|
| | medium + ATB | medium − ATB |
| Shoulder of beef meat | − | + |
| Ravioli with a Dauphin | − | ++ |
| cheese gratin | | |
| Calf liver | − | + |
| Beef kidney | − | ++ |
| Pizza dough (with baker's yeast) | + | ++ |
| Saint Marcellin - Le Canut cheese | ++ | ++ |
| Queen scallops (shellfish) | − | + |
| Mussels | − | + |
| Mung bean sprouts | ++ | ++ |
| Pecan nuts | − | + |
| Button mushrooms | − | ++ |
| Mixture of baking flours for | − | + |
| multi-cereal bread | | |

TABLE XI
Fluorescence detection by the Tempo ® Reader

| | TVC | TVC | Azo | Azo |
|---|---|---|---|---|
| | medium + | medium − | medium + | medium − |
| | ATB | ATB | ATB | ATB |
| Shoulder of beef meat | − | + | − | + |
| Ravioli with a Dauphin | + | + | − | + |
| cheese gratin | | | | |
| Calf liver | + | + | − | + |
| Beef kidney | + | + | − | + |
| Pizza dough | − | + | − | + |
| (with baker's yeast) | | | | |
| Saint Marcellin - | − | + | − | + |
| Le Canut cheese | | | | |
| Queen scallops | + | + | − | + |
| (shellfish) | | | | |
| Mussels | + | + | − | + |
| Muna bean sprouts | − | + | − | + |
| Pecan nuts | − | + | − | + |
| Button mushrooms | + | + | − | + |
| Mixture of baking flours | + | + | − | + |
| for multi-cereal bread | | | | |

Table X indicates whether there is growth on the PCA medium, thus indicating the amount of microorganism contained in a gram of matrix. The symbol “−” means <100 CFU/gram, “+” means 100<x<1 500 000 CFU/gram and “++” means >1 500 000 CFU/gram. Growth of microorganisms is observed for all the matrices incubated in a medium without antimicrobial cocktail, whereas growth of microorganisms is observed only for the pizza dough, Saint Marcellin cheese and bean sprout matrices in the presence of the antimicrobial cocktail. This phenomenon is similar to that observed in Example 3.

Table XI indicates the detection of fluorescence by the Tempo® Reader. Fluorescence is detected for all the matrices tested except for the shoulder of beef meat, the pizza dough, the Saint Marcellin cheese, the bean sprouts and the pecan nuts in the presence of the antimicrobial cocktail, whereas no fluorescence is detected under these conditions for the Azo medium. By referring to the results contained in Table X, it may be concluded that there is matrix-TVC medium interference for the ravioli with Dauphin cheese gratin, the calf liver, the beef kidney, the queen scallops, the mussels, the button mushrooms and the mixture of baking flours. This interference is not observed with the Azo medium.

For the media without microbial cocktail, growth and fluorescence are observed for all the matrices in all the media. The fluorescence detected in the Azo medium is due to the microbial growth.

These results make it possible to conclude that the “azo” compound 3-(4-hydroxyphenyl)azobenzoic acid is not degraded by the food matrices and allows the detection of the microorganisms present in these matrices.

1. Process for detecting, in a biological sample, at least one microorganism with azoreductase activity, comprising the steps consisting in:
a) placing the sample in contact with a reaction medium comprising at least one azo compound;
b) incubating the said reaction medium; and
c) detecting the reduction of the azo compound by the said microorganism, indicating the presence of the said at least one microorganism.

2. Process according to claim 1, in which step c) also enables counting.

3. Process according to claim 1, in which step c) enables the characterization of at least one group of microorganisms.

4. Process according to claim 1, in which the reduction of the azo compound is detected by measuring a variation in absorbance or fluorescence.

5. Process according to claim 4, in which step c) corresponds to the detection of disappearance of a coloration.

6. Process according to claim 4, in which step c) corresponds to the detection of appearance of a fluorescence.

7. Process according to claim 6, in which the reduction of the azo compound results in the suppression of intramolecular quenching.

8. Process according to claim 6, in which the reduction of the azo compound results in the suppression of intermolecular quenching.

9. Process according to claim 1, in which the medium comprises at least a second substrate that is capable of detecting an enzymatic reaction.

10. Process according to claim 9, in which the said second substrate is a chromogenic substrate, whose metabolism produces a coloration or a fluorescence.

11. Process according to claim 1, in which the reaction medium is a solid or liquid culture medium.

12. Process according to claim 1, wherein it is performed in a container in card form.

13. Process according to claim 12, in which the reaction card is a Vitek® or Tempo® card.

14. Process according to claim 1, in which the sample is a food matrix.

15. A method comprising: detecting at least one microorganism in a sample, with at least one azo compound.
